(19) United States
(12) Patent Application Publication
(10) Pub. No.: US 2005/0287068 A1
(43) Pub. Date: Dec. 29, 2005

(54) Efficacy of active immunotherapy by integrating diagnostic with therapeutic methods

(76) Inventors: Adrian Ion Bot, Valencia, CA (US); David C. Diamond, West Hills, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/155,928

(22) Filed: Jun. 17, 2005

Related U.S. Application Data
(60) Provisional application No. 60/580,964, filed on Jun. 17, 2004.

(51) Int. Cl. A61K 49/00; A61K 39/00

(52) U.S. Cl. 424/9.1; 424/184.1

(57) ABSTRACT

The invention described herein relates to improved strategies for designing and practicing treatments and clinical trials based upon active immunotherapy protocols, particularly by making diagnostic use of portions of the therapeutic regimen and adjusting the course of treatment if necessary. Embodiments of the invention include methods for determining a course of treatment and methods for treating a patient in which responsiveness to a non-final step of a multi-step active immunotherapy protocol is assessed to determine if, how and when to continue treatment, progress to a different stage of treatment, or discontinue treatment.

### CROSS REFERENCE TO RELATED APPLICATIONS

This application claims priority under 35 U.S.C. § 119(e) to U.S. Provisional Application No. 60/580,964, filed on Jun. 17, 2004, entitled IMPROVED EFFICACY OF ACTIVE IMMUNOTHERAPY BY INTEGRATING DIAGNOSTIC WITH THERAPEUTIC METHODS; the disclosure of which is incorporated herein by reference in its entirety.

### BACKGROUND OF THE INVENTION

1. Field of the Invention

The invention described herein relates to improved strategies for designing and practicing treatments and clinical trials based upon active immunotherapy protocols, particularly by making diagnostic use of portions of the therapeutic regimen and adjusting the course of treatment if necessary.

2. Description of the Related Art

The standard practice in both clinical practice and clinical trial design is to carry out diagnostic tests, specify a treatment protocol, and evaluate the effectiveness of the treatment in retrospect, at some point following the completion of the protocol. Especially for less well understood or complex therapies, such as active immunotherapy, this type of protocol can lead to an extended period of time in which a subject is treated with agents that will never work for this patient. In addition, active immunotherapy based on fixed protocols may be inappropriate for some patients. In trials this leads to increased costs, an obscuring of positive results, a need for larger trial populations, and an associated increase in the length of clinical trials before a reliable answer can be obtained. Clinically this leads to the purposeless consumption of expensive products and lost opportunity for some patients to pursue other, potentially better suited, treatment options. Thus, it is an object of the present invention to allow for the adjustment of the course of treatment in terms of continuance, dosage, and frequency of administration and the like, to the response profile of the patient so as to optimize the overall beneficial effect. There is a need for active immunotherapy treatments that are allowed to evolve in response to a patient's immune response.

### SUMMARY OF THE INVENTION

Embodiments of the invention described herein include methods for determining a course of treatment in which responsiveness to a non-final step of a multi-step active immunotherapy protocol is assessed to determine if, how and when to continue treatment, progress to a different stage of treatment, or discontinue treatment.

In one embodiment, the disclosed methods can include the steps of administering to a patient an immunogenic composition as part of a non-final step of a multi-step immunotherapy protocol; measuring an immune response in the patient subsequent to the non-final step; and selecting a subsequent treatment action based on the measurement.

In some embodiments of the invention, the immunogenic composition comprises a target-matched immunogen. In other embodiments, the immunogen comprises an antigen or a portion thereof. The immunogenic composition can further comprise an immunopotentiating agent. In still other embodiments, the immunogen comprises a nucleic acid encoding the antigen or portion thereof. In some embodiments, the immunogenic composition is multivalent.

The methods disclosed herein can be used with any multi-step active immunotherapy protocol, such as, for example, prime-boost, induce-and-amplify, or entrain-and-amplify protocols. These protocols are used throughout as exemplary protocols. Other similar protocols for use with the methods described herein will be apparent to those of skill in the art.

In some embodiments, the methods are applied to a prime-boost protocol in which the protocol calls for at least one priming dose. In some embodiments, the protocol calls for two, or three, or four, or five, or six, or more priming doses. In some embodiments, the priming dose (or doses) is followed by at least one boosting dose. In some embodiments, the protocol calls for two, or three, or four, or five, or six, or more boosting doses. In some embodiments, the protocol calls for the prime-boost cycle to be repeated one or more times. In one embodiment, the priming dose(s) is a plasmid encoding an immunogenic polypeptide. Alternatively, the priming dose(s) is an immunogenic polypeptide plus an immunopotentiating agent. The agent can be, for example, a toll-like receptor ligand, endocytic-Pattern Recognition Receptor (PRR) ligands, quillaja saponins, tucaresol, and cytokines, or any other agent that activates innate immunity. Preferably, the doses are delivered directly to the lymphatic system. In particularly preferred embodiments, the doses are delivered directly to a lymph node or lymph vessel. Delivery can be by injection or infusion.

In one embodiment, the methods are applied to an induce-and-amplify protocol in which the protocol calls for at least one inducing dose. In some embodiments, the protocol calls for two, or three, or four, or five, or six, or more inducing doses. In some embodiments, the inducing dose (or doses) is followed by at least one amplifying dose. In some embodiments, the protocol calls for two, or three, or four, or five, or six, or more amplifying doses. In some embodiments, the protocol calls for the induce-and-amplify cycle to be repeated one or more times. In one embodiment, the inducing dose(s) is a plasmid encoding an immunogenic polypeptide. Alternatively, the inducing dose(s) is an immunogenic polypeptide plus an immunopotentiating agent. The agent can be, for example, a toll-like receptor ligand, endocytic-Pattern Recognition Receptor (PRR) ligands, quillaja saponins, tucaresol, and cytokines, or any other agent that activates innate immunity. Preferably, the doses are delivered directly to the lymphatic system. In particularly preferred embodiments, the doses are delivered directly to a lymph node or lymph vessel. Delivery can be by injection or infusion.

In another embodiment, the methods are applied to an entrain-and-amplify protocol in which the protocol calls for at least one entrainment dose. In some embodiments, the protocol calls for two, or three, or four, or five, or six, or more entraining doses. In some embodiments, the entraining dose (or doses) is followed by at least one amplifying dose. In some embodiments, the protocol calls for two, or three, or four, or five, or six, or more amplifying doses. In some embodiments, the protocol calls for the entrain-and-amplify cycle to be repeated one or more times. In one embodiment, the entraining dose(s) is a plasmid encoding an immunogenic polypeptide. Alternatively, the entraining dose(s) is an immunogenic polypeptide plus an immunopotentiating agent. The agent can be, for example, a toll-like receptor ligand, endocytic-Pattern Recognition Receptor (PRR) ligands, quillaja saponins, tucaresol, and cytokines, or any other agent that activates innate immunity. Preferably, the doses are delivered directly to the lymphatic system. In particularly preferred embodiments, the doses are delivered directly to a lymph node or lymph vessel. Delivery can be by injection or infusion.

In some embodiments, an immune response is measured or evaluated subsequent to a non-final step in the immunotherapy protocol. Thus, in some embodiments, the methods are applied using an induce-and-amplify protocol and the immune response is measured after the first, second, third or more or final inducing dose. In other embodiments, the methods are applied using an induce-and-amplify protocol and the immune response is measured after a non-final amplifying dose. In other embodiments, a prime-boost protocol is used and the immune response is measured after the first, second, third or more or final priming dose. In another embodiment, a prime-boost protocol is used and the immune response is measured after a non-final boosting dose. In still other embodiments, the methods are applied to an entrain-and-amplify protocol and the immune response is measured after the first, second, third or more or final entraining dose. In other embodiments, an entrain-and-amplify protocol is used and the immune response is measured after a non-final amplifying dose. In some embodiments, the immune response is measured at a single time point in the protocol. In other embodiments, the immune response is measured at multiple time points in the protocol. In some embodiments, the method includes at least two assaying steps carried out at different time points during the course of treatment, wherein comparative information is obtained from the assaying steps. The obtained information can be used to implement, modify or withdraw a therapy. In some embodiments, the first of the at least two assaying steps is carried out prior to commencement of the treatment to establish a baseline immunity. The immune response can be measured 1, or 2, or 3, or 4, or 5, or more times during the course of treatment. In still other embodiments, the immune response can be measured continuously, e.g., intermittently throughout the course of treatment, or after every non-final step of the protocol. Thus, the non-final dose of the protocol serves a dual role of a therapeutic and a diagnostic.

In some embodiments, evaluation of immune responsiveness can be assessed, for example, by an Elispot assay, preferably, an antigen-specific Elispot analysis, or flow cytometry staining with MHC-multimers. In other embodiments, immune responsiveness can be assessed, for example, by a DTH assay, preferably for an antigen-specific DTH, antibody assays or, 1° or 2° cytotoxicity assays. In still other embodiments, immune responsiveness can be measured using a cytokine assay, a cell proliferation assay, a chromium release assay, an immunofluorescence assay, and an inflammatory reaction assay. Additional assays will be readily apparent to those of skill in the art.

In some embodiments, the subsequent course of treatment action can include, for example, administering a subsequent dose as called for in the protocol, adjusting the protocol, or discontinuing treatment prior to completion of the protocol. In some embodiments, adjusting the protocol includes, for example, but not limited to, administering a subsequent dose of the protocol, administering a subsequent dose at an increased dosage, administering a subsequent dose at a decreased dosage, administering subsequent doses more frequently, administering subsequent doses less frequently, repeating administration of said preceding dose, selectively administering individual components of the composition, selectively suspending administration of individual components of the composition, and discontinuing treatment prior to completion of the protocol.

For example, in some embodiments, the measuring step indicates no immune response, and the selecting step includes discontinuation of the immunotherapy protocol. In other embodiments, the measuring step indicates a minimal immune response and the selecting step includes repeating the non-final dose of the protocol. In one embodiment, a marginal or no antigen-specific immune response is detected after a non-final dose and the non-final dose is repeated. In some embodiments, repeating the non-final dose can further entail a schedule/frequency and/or dosage adjustment to increase and maintain the immune response as desired. In another embodiment, a marginal or no antigen-specific immune response is detected after the non-final dose and treatment is discontinued prior to completion of the protocol. In still another embodiment, a significant antigen-specific immune response is detected and treatment is continued according to the protocol. Alternatively, treatment is continued according to an altered protocol. For example, the schedule/frequency and/or dosage of subsequent doses can be increased or decreased, or subsequent doses or steps can be selectively repeated or skipped, or individual components of the compositions of subsequent doses or steps can be selectively administered or suspended.

In some embodiments, a dosage form that is different than the non-final dose is administered. For example, a boosting dose can be administered, comprising the use of a virus or viral vector as the different dosage form. Alternatively, an amplifying dose can be used, comprising the use of an intralymphatically delivered peptide as the different dosage form. In one embodiment, the peptide is free of adjuvant.

In other embodiments, the measuring step indicates a substantial immune response and the selecting step includes administering a second immunogenic composition. The immunogenic compositions can be provided in a form selected from the group consisting of DNA, mRNA, plasmid, peptide, polypeptide, protein, viral vector, virus-like particle, and bacterial vector. In some embodiments, the first and second immunogenic compositions are provided in a form that is the same. In other embodiments, the first and subsequent immunogenic compositions are provided in forms that are different.

In some embodiments a multivalent immunogenic composition(s) is used. In some embodiments, the multivalent composition comprises at least two target antigens. In some embodiments, the multivalent composition comprises at least three, four, five, or more target antigens. In such cases, the measurement of the immune response can be carried out by multiple methods against a panel of antigens corresponding to or encompassing those targeted by the multivalent composition(s). In some embodiments, if a significant response is measured against a first antigen, etc. but no response is detected against a second antigen, etc., after a non-final dose, subsequent treatment can be adjusted accordingly. In some embodiments, subsequent treatment can be focused on the antigen or antigens against which a response was detected, for example, by discontinuing administration of the component targeting the antigen against which no response was detected.

In still another embodiment, if subsequent to a non-final step of the protocol, an immune response against certain components of the immunogenic composition is detected, but is not significant, or is suboptimal (e.g., below a preset threshold value), the protocol can be modified to compensate, for example, the subsequent treatment action can include providing the subdominant components in greater amount or more frequently.

In still another embodiment, if the immune response detected against one or multiple components indicates immune tolerance, then the protocol is modified, for example, by subtracting such components.

Still other embodiments provide a method of treating a patient, wherein the method includes sequentially the steps of: administering to the patient an immunogenic composition as part of a non-final step of a multi-step immunization protocol; assaying a patient sample for immune responsiveness to a component of the composition subsequent to the non-final step; classifying the patient as a responder, a low-responder, or a non-responder based on the immune responsiveness; and selecting a subsequent treatment action based on the classification.

For example, in some embodiments, the classifying step comprises classifying the patient as a non-responder and the selecting step comprising discontinuing treatment.

In some embodiments, the non-final dose is an inducing dose of an induce-and-amplify protocol. In some embodiments, the classifying step comprises classifying the patient as a low-responder and the selecting step comprising administering an additional inducing dose. In other embodiments, the classifying step comprises classifying the patient as a responder and the selecting step comprising administering an amplifying dose.

In some embodiments, the immunogenic composition comprises a composition targeting antigens. In such embodiments, the assaying step can include determining immune responsiveness to at least two target antigens. The classifying step can include classifying the patient as a responder with respect to a first target antigen and a low-responder with respect to a second target antigen, and the selecting step comprises administering an immunogenic composition comprising a component corresponding to the second target antigen, but not to the first target antigen.

Yet other embodiments relate to a method of treating a patient comprising the steps of: administering to the patient an immunogenic composition as part of a non-final step of a multi-step immunotherapy protocol; wherein the immunogenic composition targets one or more antigens; assaying tumor tissue from the patient for expression of the one or more antigens subsequent to the non-final step; establishing an antigen expression profile; and optimizing the match between the expression profile and the one or more antigens targeted by the immunogenic composition.

Other embodiments relate to the design and conduct of clinical trial programs for active immunotherapies. In some embodiments pre-existing and/or treatment-induced immune reactivity can be used to stratify a patient population based on clinical outlook. In one embodiment, immunogenicity and/or immune responsiveness can be evaluated in a patient population, which is then stratified into subpopulations, such as “non-responders,” “responders,” “low responders,” “high responders,” and the like, or other similar classifications or categories that correspond to the level of immune responsiveness detected. Effectiveness of the treatment can then be separately evaluated in the two (or more) subpopulations. In one embodiment, effectiveness is evaluated only in the responder subpopulation. In one embodiment, effectiveness is not evaluated in the non-responder subpopulation, but is evaluated separately in low responder and high responder subpopulations. In one embodiment, treatment is discontinued for subpopulations that are not evaluated for efficacy.

Some embodiments relate to a method of determining the responsiveness of a patient to immunotherapy with a substance X, wherein the method includes the steps of assaying a blood sample from said patient immunized with the substance X for immune responsiveness by determining the number of cytotoxic T lymphocytes (CTL); and classifying the patient as a “responder”, “non-responder” or “low responder” on the basis of the number of CTL.

### BRIEF DESCRIPTION OF THE DRAWINGS

FIGS. 1A and 1B show a positive correlation of staining for antigen specific T cell receptor and cytolytic activity in a preclinical model, following a two-phase immunization protocol.

FIGS. 2A-C show a correlation between the magnitude of immune response as measured by tetramer staining and clearance of human tumor cells in vivo, in a preclinical model.

FIG. 3 shows that a multivalent immune response (as measured by ELISPOT analysis) correlates with in vivo clearance of human tumor cells, in contrast with a monovalent response, in a preclinical model.

FIG. 4 shows that certain immune active molecules have an inverse dose effect relationship.

FIG. 5 shows an immunization protocol in a preclinical model, in which multivalent priming against dominant and subdominant antigens is followed by boost with the subdominant antigens, to induce a balanced response.

FIGS. 6A and 6B show heterogeneity of immune response in a preclinical model using the multivalent immunization protocol.

FIGS. 7A and 7B show that by selective boosting with subdominant epitopes, a balanced, multivalent immune response can be achieved in a preclinical model.

FIG. 8 depicts a clinical study aimed at analyzing the relationship between immune reactivity against a tumor associated antigen and clinical outlook.

FIG. 9 shows that patients that are immune reactive against Melan-A (pre- or post-treatment) have a significantly increased time to disease progression.

FIG. 10 shows schema for integrated therapeutic and diagnostic practice.

FIG. 11 is an exemplary decision tree showing how the general principles described herein can be applied to a particular protocol.

### DETAILED DESCRIPTION OF THE PREFERRED EMBODIMENT

Described herein are methods to monitor and adjust treatment for cancer, inflammatory or infectious diseases, wherein the treatment is based upon an active immunotherapy protocol, and wherein the methods are applied after the initiation of treatment and before the completion of the protocol. Such methods allow the adjustment, continuation or termination of immunotherapeutic protocols in a way that optimizes the treatment. This flexibility and customizability improves the success rate of immunotherapy and overall outcome.

The methods disclosed herein are facilitated by the use of potent immunogenic compositions with dual roles: (1) to initiate or maintain or enhance a therapeutic effect, and (2) to allow for the reliable assessment of a patient's immune response to a component or components of the compositions prior to completion of the treatment protocol. Such coupling of diagnostic and therapeutic methods is based, in part, on research-based evidence demonstrating a correlation between magnitude of immune response and effector function (Example 1).

The benefits of the disclosed methods include: (1) improving the overall efficacy of an active immunotherapy protocol by adjusting the course of treatment called for in the protocol based on reliable assessments of each patient's immune response prior to a non-final step of the protocol; (2) identifying patients with the highest potential to benefit from a particular immunotherapy protocol after the treatment has been initiated and prior to a non-final step of the protocol; (3) increasing the size of the treated population that would likely benefit from a given immunotherapy protocol by avoiding or minimizing the decisional input of less precise enrollment or exclusion criteria (e.g., decisional input based on DTH to an unrelated antigen, blood cell counts, or previous/concurrent treatments such as chemotherapy that may be compatible with immunotherapy); (4) improving the quality of life of cancer patients or chronically infected patients by discontinuing treatment unlikely to be beneficial; (5) increasing the life span of cancer patients unlikely to benefit from a particular treatment protocol by timely enrolling non-responders in alternative, more appropriate therapies; (6) reducing health care/treatment costs by minimizing the ineffective use of expensive biotherapeutics in general; (7) determining a course of treatment that is tailored to each individual patient; and (8) increasing the quality of active immunotherapy treatments.

Definitions

As used herein, “active immunotherapy” refers to attempts to stimulate the body's own immune system to fight the disease. In some embodiments the therapeutic effect is mediated by a cytolytic T cell (CTL) response. In other embodiments other types of immune responses, including, for example, antibody, T helper, and T regulatory responses mediate the therapeutic effect, alone or in any combination. In some cases it is desirable to generate one type of response in the absence of another type, for example, to generate a CTL or antibody response in the absence of a T helper and/or T regulatory response.

As used herein, “treatment protocol” or “protocol” refers to a plan for a medical treatment or an ideal course of treatment. The treatment protocols or protocols for use in the methods described herein are therapeutic regimens for use in clinical or medical settings.

As used herein, “treatment” refers to the act of treating a patient medically, such as by administration or application of remedies to a patient.

As used herein, “non-final step” refers to the non-final step of a protocol or pre-established plan for treatment. This is distinguishable from a non-final step of the treatment of a patient. In some embodiments of the invention, a non-final step of a protocol can be a final step of the treatment.

Measuring the Immune Response

In embodiments of the methods disclosed herein, the subsequent course of treatment of a patient being treated according to an immunotherapy protocol is determined using methods to rapidly and reliably assess a patient's immune response to a component or multiple components of an immunogenic composition that is administered as a non-final dose of the protocol.

In some embodiments, a patient sample, such as blood, or other bodily fluids, or secretions, or portions thereof, such as lymphocytes or cytokines, is assayed for an immune response. In some embodiments, the immune response is measured using visual observations of the body, such as a skin test for DTH.

In some embodiments, only the desired response(s) is assayed for. In other embodiments, only the undesired response(s) is assayed for. In still other embodiments, both the desired and undesired responses are assayed for. When assaying for undesired responses, the classification of patients and/or subsequent treatment actions are the reverse of those described below. For example, in some embodiments wherein tissue samples are assayed for undesired responses, for patients in whom no immune response is detected, e.g., “non-responders” treatment is continued according to protocol and for patients in whom a significant immune response is detected, e.g., “responders” treatment is discontinued.

Assay Technology

Exemplary assessment methods include, for example, but not limited to, tetramer-based T cell staining, ELISPOT analysis, flow cytometry staining with MHC-multimers, DTH assay, preferably for an antigen-specific DTH, antibody assays, 1° or 2° cytotoxicity assays, cytokine assays, cell proliferation assays, chromium release assays, immunofluorescence assays, or inflammatory reaction assays, all of which are well known in the art. Several of these methodologies are utilized in the examples below. Additional assays will be readily apparent to those of skill in the art.

In some embodiments it can be advantageous to assess tumor antigen expression. Many technologies to carry out such assays are known in the art. Tumor tissue or fragments, including tumor antigens, to assay can be obtained as bulk tissue through surgery or in cellular form from blood, bone marrow, cell aspirates, peritoneal lavage, plural aspirates, or bronchial washes, and the like.

Generally, any reliable method of detecting specific proteins or mRNAs can be adapted. Preference is given to techniques based on characteristics such as the ability to assay large numbers of samples and/or provide results quickly or that the assay is inexpensive to practice, or some optimum of these parameters. Commonly, detection of specific proteins involves the use of antibodies. Immunohistochemistry (IHC) is broadly applicable, but western hybridization, radioimmunoassay (RIA), and flow cytometry can also be used; collectively protein determinations. TRC-tetramers and antibodies recognizing specific peptide-MHC complexes can also be used. Tumor tissue can be used as target or stimulator in a wide variety of immunological assays (Elispot, T cell hybridoma reactivity, microcytotoxicity, and the like). Such assays are specific for a target epitope, not just the parent antigen, and thus can be referred to as epitope determinations. Detection of specific mRNA can be accomplished using any of several modalities of RT-PCR (reverse transcription-polymerase chain reaction) and similar nucleic acid amplification techniques (e.g., 3SR), northern hybridization, querrying of gene arrays with mRNA or cDNA, and in situ hybridization; collectively transcript determinations. Reagents that detect presentation of particular T cell epitopes from target antigens can also be used. These include, for example, T cell lines and hybridomas, and more preferably, antibodies specific for the peptide-MHC complex and TCR tetramers (see for example Li et al. Nature Biotech. 23:349-354, 2005 which is incorporated herein by reference in its entirety).

PCR techniques are sensitive and generally easy to implement, however they cannot detect the mosaicism of antigen expression within a sample. IHC (and other in situ techniques), though potentially more labor intensive, allow spatial variation of expression within a sample to be observed. Thus distinctions between co-expression of antigens within the same cells versus co-expression within different cells within the same sample can be made. Both situations can be desirable, the former providing for greater redundancy of targeting and reduced likelihood of antigen-loss escape mutants arising, the latter revealing how a greater proportion of the total tumor tissue can be directly targeted. Such information is also relevant to the use of antigens with more complex expression patterns. For example, PSMA, which can be expressed by prostate cells and tumor neovasculature, can be used as a prostate lineage marker if its expression can be associated specifically with the neoplastic cells, either through use of an in situ detection methodology or microdissection before assaying expression.

Stratification of Patients

In some embodiments, patients are classified according to whether an immune response is detected following a non-final dose of the protocol. For example, in some embodiments, each patient is classified as a “non-responder,” “low responder,” or other similar classification, based on his or her detected immune response in relation to predetermined values. Thus, in some embodiments, patients whose immune response falls below a predetermined low value are categorized or classified as “non-responders,” while patients whose immune response falls above a predetermined low value but below a predetermined optimal value are classified as “low responders,” and patients whose immune response falls above a predetermined optimal value are classified as “responders.” As will be apparent to those of skill in the art, the predetermined values are dependent upon the technique used to measure the immune response and the response being sought. These values will be apparent to those of skill in the art for a particular type of assay or measurement, as will additional or alternate classifications/stratifications useful for the methods described herein.

Subsequent Treatment Actions

In some embodiments, the subsequent course of treatment action can include, for example, administering a subsequent dose as called for in the protocol, adjusting the protocol, or discontinuing treatment prior to completion of the protocol. In some embodiments, adjusting the protocol includes, for example, but not limited to, administering a subsequent dose (or doses) at an increased dosage, administering a subsequent dose (or doses) at a decreased dosage, administering subsequent doses more frequently, administering subsequent doses less frequently, repeating administration of the non-final dose, selectively repeating or skipping subsequent doses, selectively administering individual components of the immunogenic composition, and/or selectively suspending administration of individual components of the composition.

In some embodiments, patients tagged as “responders” after a non-final step in the immunotherapy protocol continue treatment according to protocol while patients tagged as “non-responders” discontinue treatment according to the protocol and can be subsequently enrolled in alternative therapeutic regimens. In some embodiments, patients identified as “low-responders” continue treatment according to an altered protocol that can include, for example additional, more frequent, or increased doses of the therapeutic agent.

Thus, treatment protocols can be adjusted based on the responsiveness to induction or amplification phases and variation in antigen expression. For example, rather than amplifying after some set number of entrainment doses, repeated entrainment doses can be administered until a detectable response is obtained, and then amplifying peptide dose(s) can be administered. Similarly, scheduled amplifying or maintenance doses of peptide can be discontinued if their effectiveness wanes, antigen-specific regulatory T cell numbers rise, or some other evidence of tolerization is observed, and further entrainment can be administered before resuming amplification with the peptide.

Continued Monitoring

The disclosed diagnosis-therapy combination is also useful in scenarios where disease progression is detected after an initial favorable response to immunotherapy. There are many mechanisms for tumors to escape immune attack, for example, loss of expression of TuAAs or HLA. Thus, when an existing tumor resumes growth or a new metastasis is detected, a tissue sample can be analyzed (such as for expression of TuAAs and HLA, sensitivity to chemotherapeutic agents, etc.). Depending on the results of the analysis, appropriate and effective therapy for the new (or mutated) tumor can be initiated. For example, if TuAAs corresponding to the applied immunogenic composition are no longer expressed by the new tumor but other TuAAs are expressed, then immunogenic compositions containing appropriate antigens can be used to treat the newly discovered or mutated tumor.

In some embodiments, the disclosed diagnosis-treatment cycles are repeated throughout the protocol to monitor the ongoing status of the patient and respond to any changes in the immune response. The continued use of diagnosis-treatment cycles ensures that effective treatment methods are applied at all times thus, maximizing the quality of life for the patient while decreasing treatment or clinical trial costs by minimizing the administration of inappropriate therapy. In addition, the use of diagnosis-treatment cycles affords the opportunity for patients tagged “non-responder” to receive alternative and more appropriate forms of therapy.

Active Immunotherapy Protocols

The principles of the methods disclosed herein are applicable to protocols for active immunotherapy generally. They are well suited to protocols that utilize an initial dosage form that establishes the immune response and a second dosage form that intensifies the response to clinically effective levels. An example of such an approach is a “prime-boost” protocol involving an initial immunization dose or doses with a nucleic acid composition encoding the immunogen, most typically a naked or lipid complexed DNA plasmid and a booster immunization dose or doses using a viral vector. See for example, U.S. Pat. No. 6,663,871 entitled “METHODS AND REAGENTS FOR VACCINATION WHICH GENERATE A CD8 T CELL IMMUNE RESPONSE,” which is hereby incorporated by reference in its entirety. Another exemplary approach utilizes entrain-and-amplify protocols, such as those disclosed in U.S. patent application Ser. No. 10/871,707, filed on Jun. 17, 2004 (Publication No. 20050079152 A1) and U.S. Provisional Application No. 60/640,402, filed Dec. 29, 2004, both entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSE,” each of which is hereby incorporated by reference in its entirety. Another exemplary approach is disclosed in U.S. Provisional Application No. 60/640,821, filed Dec. 29, 2004.

For example, in some embodiments, the treatment protocols call for injection or infusion into one or more lymph nodes, starting with a number (e.g., 1 to 10, or more, 2 to 8, 3 to 6, preferred about 4 or 5) of administrations of recombinant DNA (dose range of 0.001-10 mg/kg, preferred 0.005-5 mg/kg) followed by one or more (preferred about 2) administrations of peptide, preferably in an immunologically inert vehicle or formulation (dose range of 1 ng/kg-10 mg/kg, preferred 0.005-5 mg/kg). Because dose does not necessarily scale linearly with the size of the subject, doses for humans can tend toward the lower, and doses for mice can tend toward the higher, portions of these ranges. In some embodiments, the preferred concentration of plasmid and peptide upon injection is generally about 0.1 μg/ml-10 mg/ml, and the most preferred concentration is about 1 mg/ml, generally irrespective of the size or species of the subject. However, particularly potent peptides can have optimum concentrations toward the low end of this range, for example between 1 and 100 μg/ml. When peptide only protocols are used to promote tolerance doses toward the higher end of these ranges are generally preferred (e.g., 0.5-10 mg/ml). This sequence can be repeated as long as necessary to maintain a strong immune response in vivo. Moreover, the time between the last entraining dose of DNA and the first amplifying dose of peptide is not critical. Preferably it is about 7 days or more, and can exceed several months. The multiplicity of injections of the DNA and/or the peptide can be reduced by substituting infusions lasting several days (preferred 2-7 days). It can be advantageous to initiate the infusion with a bolus of material similar to what might be given as an injection, followed by a slow infusion (24-12000 μl/day to deliver about 25-2500 μg/day for DNA, 0.1-10,000 μg/day for peptide). This can be accomplished manually or through the use of a programmable pump, such as an insulin pump. Such pumps are known in the art and enable periodic spikes and other dosage profiles, which can be desirable in some embodiments.

Exemplary epitopes and epitope analogues are described in U.S. patent application Ser. Nos. 10/117,937, filed Apr. 4, 2002, (Pub. No. 20030220239 A1); Ser. Nos. 11/067,159 and 11/067,064, each filed Feb. 25, 2005; U.S. patent application Ser. No. 10/657,022 filed Sep. 5, 2003, and PCT Application Nos. PCT/US2003/027706, filed Jun. 17, 2004 (Pub. No. WO 04022709 A2) and PCT/US02/11101, filed Apr. 4, 2002 (Pub. No. WO 02081646), all entitled “EPITOPE SEQUENCES”; and U.S. patent application Ser. No. 10/292,413, filed Nov. 7, 2002 (Pub. No. 20030228634 A1) entitled “EXPRESSION VECTORS ENCODING EPITOPES OF TARGET-ASSOCIATED ANTIGENS AND METHODS FOR THEIR DESIGN”; U.S. Provisional Application No. 60/581,001, filed Jun. 17, 2004, and U.S. Patent Application No. __/______, filed on the same date as the instant application (Attorney Docket: MANNK.038A), entitled “SSX-2 PEPTIDE ANALOGS”; U.S. Provisional Application No. 60/580,962, filed on Jun. 17, 2004, and U.S. Patent Application No. __/______, filed on the same date as the instant application (Attorney Docket MANNK.039A), filed on the same date as the instant application, entitled “NY-ESO-1 PEPTIDE ANALOGS;” and U.S. Patent Application No. __/______, filed on the same date as the instant application (Attorney Docket MANNK.051A), filed on the same date as the instant application, and U.S. Provisional Patent Application No. __/______, filed on the same date as the instant application (Attorney Docket MANNK.052PR), filed on the same date as the instant application, both entitled EPITOPE ANALOGS, each of which is hereby incorporated by reference in its entirety.

Particularly useful combinations of tumor-associated antigens for immunotherapeutics directed against specific tumors are disclosed in U.S. Provisional Patent Application No. 60/479,554, filed on Jun. 17, 2003, and U.S. patent application Ser. No. 10/871,708, filed Jun. 17, 2004, and U.S. Provisional Patent Application No. 60/640,598, filed Dec. 29, 2004, all entitled “COMBINATIONS OF TUMOR-ASSOCIATED ANTIGENS IN VACCINES FOR VARIOUS TYPES OF CANCERS,” and U.S. Provisional Patent Application No. 60/580,969, filed Jun. 17, 2004, and U.S. Patent Application No. __/______, filed on the same date as the instant application (Attorney Docket No: MANNK.050A), both entitled “COMBINATIONS OF TUMOR-ASSOCIATED ANTIGENS IN DIAGNOSTICS FOR VARIOUS TYPES OF CANCER,” and U.S. Provisional Application No. __/______ (Atty Docket No. MANNK.054PR), entitled MULTIVALENT ENTRAIN-AND-AMPLIFY IMMUNOTHERAPEUTICS FOR CARCINOMA, filed on date even with this disclosure, each of which is also hereby incorporated by reference in its entirety. Nucleic acid vectors for expressing epitopes that can be used in embodiments of the invention described herein, and methods for their design are disclosed in U.S. patent application Ser. No. 09/561,572, filed on Apr. 28, 2000, Ser. No. 10/225,568, filed on Aug. 20, 2002 (Pub No. 20030138808), and PCT Application No. PCT/US2003/026231, filed on Aug. 19, 2003 (Pub. No. WO 2004/018666), all entitled “EXPRESSION VECTORS ENCODING EPITOPES OF TARGET-ASSOCIATED ANTIGENS”; U.S. patent application Ser. Nos. 10/026,066, filed on Dec. 7, 2001 (Pub. No. 20030215425 A1), 10/895,523, filed on Jul. 20, 2004, Ser. No. 10/896,325, filed on Jul. 20, 2004, all entitled “EPITOPE SYNCHRONIZATION IN ANTIGEN PRESENTING CELLS”; and U.S. patent application Ser. Nos. 10/292,413, filed on Nov. 7, 2002 (Pub. No. 20030228634 A1), 10/777,053, filed on Feb. 10, 2004 (Pub No. 20040132088 A1) and 10/837,217, filed on Apr. 30, 2004, all entitled “EXPRESSION VECTORS ENCODING EPITOPES OF TARGET-ASSOCIATED ANTIGENS AND METHODS FOR THEIR DESIGN”; U.S. patent application Ser. Nos. 10/094,699, filed Mar. 7, 2002 (Pub. No. 20030046714 A1) and Ser. No. 11/073,347, filed Mar. 4, 2005, both entitled “ANTI-NEOVASCULATURE PREPARATIONS FOR CANCER”; U.S. Pat. No. 6,861,234, entitled “METHOD OF EPITOPE DISCOVERY”; U.S. patent application Ser. No. 09/561,571, filed Apr. 28, 2000 entitled “EPITOPE CLUSTERS”; U.S. patent application Ser. No. 09/560,465, filed Apr. 28, 2000, U.S. patent application Ser. No. 10/026,066, filed Dec. 7, 2001 (Pub. No. 20030215425 A1), 10/895,523, filed Jul. 20, 2004, Ser. No. 10/896,325, filed Jul. 20, 2004 entitled “EPITOPE SYNCHRONIZATION IN ANTIGEN PRESENTING CELLS”; and U.S. Pat. No. 6,709,844, and U.S. patent application Ser. No. 10/437,830, filed May 13, 2003 (Pub. No. 20030180949 A1), both entitled “AVOIDANCE OF UNDESIRABLE REPLICATION INTERMEDIATES IN PLASMID PROPAGATION,” and U.S. Provisional Application No. __/______ (Atty Docket No. MANNK.053PR), entitled METHODS AND COMPOSITIONS TO ELICIT MULTIVALENT IMMUNE RESPONSES AGAINST DOMINANT AND SUBDOMINANT EPITOPES, EXPRESSED ON CANCER CELLS AND TUMOR STROMA, filed on date even with this disclosure, each of which is hereby incorporated by reference in its entirety.

Intranodal administration for the generation of CTL and appropriate assays for detecting the response are taught in U.S. patent application Ser. No. 09/380,534, filed Sep. 1, 1999 and 09/776,232, filed Feb. 2, 2001 (Pub. No. 20020007173 A1), and in PCT Application No. PCT U.S. 98/14289 (Pub. No. WO9902183A2) each entitled “A METHOD OF INDUCING A CTL RESPONSE,” and U.S. Application No. 60/640,727, filed Dec. 29, 2004, entitled METHODS TO TRIGGER, MAINTAIN AND MANIPULATE IMMUNE RESPONSES BY TARGETED ADMINISTRATION OF BIOLOGICAL RESPONSE MODIFIERS INTO LYMPHOID ORGANS,” each of which is hereby incorporated by reference in its entirety.

Each of the applications referenced herein is hereby incorporated by reference in its entirety.

The foregoing paragraphs are intended to illustrate how the general concepts of the invention can be applied in practice and are not to be taken as an exhaustive or limiting recitation of the possible variations. Indeed many further variations will be suggested by the properties of particular immunogenic compositions and immunization protocols, and will become apparent to those of skill in the art. Individual embodiments can specifically include or exclude any such alternatives.

### EXAMPLES

The following examples relate to active immunotherapy based on the generation of cytolytic T cells. However, the underlying principles exemplified are also generally applicable to immunotherapeutics designed to generate other types immune response, including antibody, T helper, and T regulatory responses, alone or in any combination, as will be apparent to one of skill in the art.

### Example 1

### Correlation Between in Vivo Effector Function and Magnitude of Immunity was Assessed by Tetramer Staining in MHC Transgenic Mice Immunized with a Potent Regimen Encompassing Inducing with Plasmid and Amplifying with Peptide

To evaluate the immune response obtained by an entrain-and-amplify protocol, a group of immunized animals (n=7) was challenged with melan-A peptide (ELAGIGILT V (SEQ ID NO: 1)) loaded target cells in vivo. Splenocytes were isolated from littermate control HHD mice and incubated with 20 μg/mL peptide for 2 hours. These cells were then stained with CFSE^{hi} fluorescence (4.0 μM for 15 minutes) and intravenously co-injected into immunized mice with an equal ratio of control splenocytes stained with CFSE^{lo} fluorescence (0.4 μM). Eighteen hours later the specific elimination of target cells was measured by removing spleen, lymph node, PBMC, and lung from challenged animals and measuring CFSE fluorescence by flow cytometry.

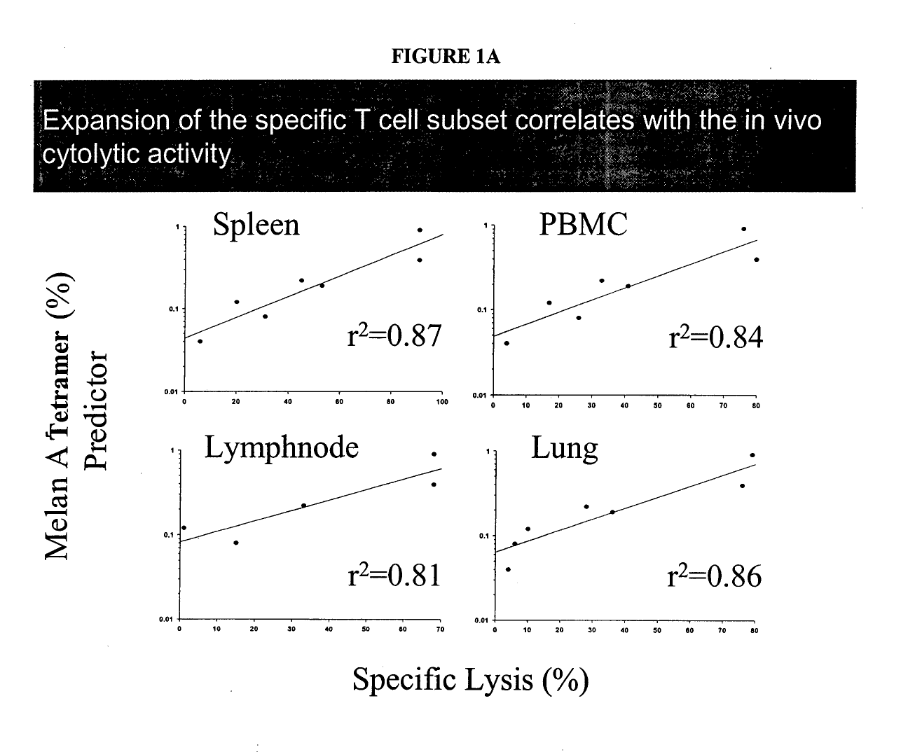

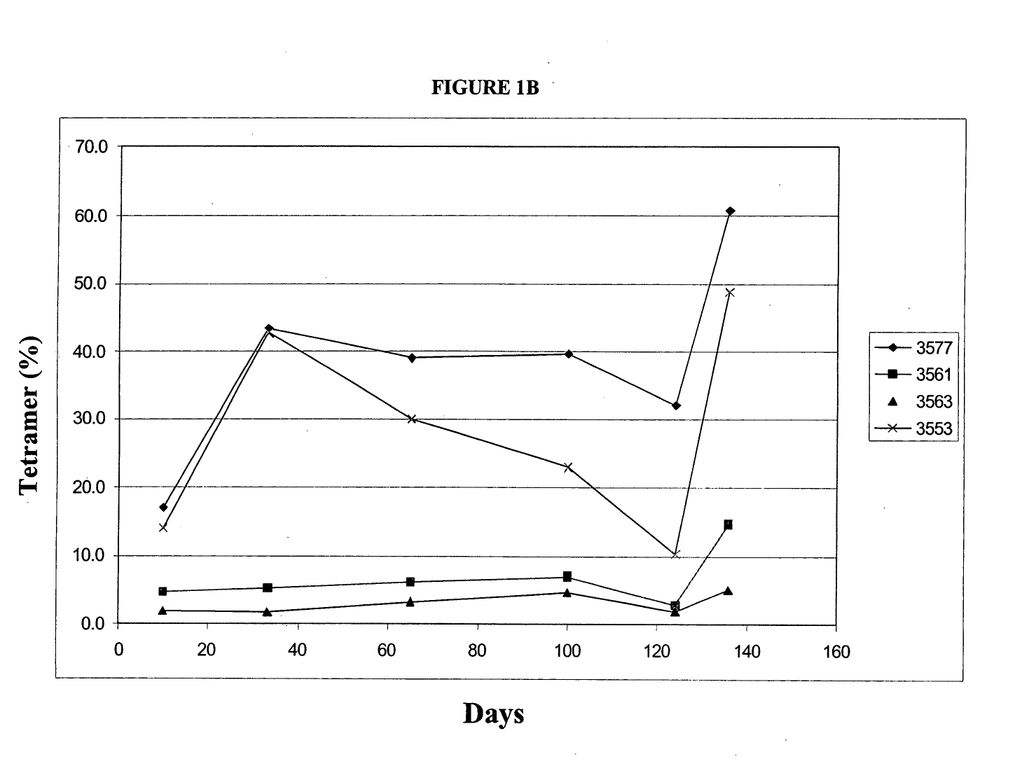

The mice demonstrated high levels of specific killing of target cells in lymphoid as well as non-lymphoid organs and demonstrated a specific correlation with tetramer levels (See FIG. 1A, r²=0.81 or higher for all tissues tested). Thus, tetramer staining was a reliable indication of the generation of cytolytic T cells using this immunization protocol.

Mice immunized using a prime boost protocol as above (plasmid on days 1, 4, 15, 19 and peptide on days 28 and 32), received an additional therapeutic cycle-essentially a prime boost repeat, on days 46, 49, 60, 64 (plasmid), 74 and 78 (peptide) respectively. This was followed by yet another peptide boost to assess immune memory, on day 125. The magnitude of immune response was monitored during this interval, by tetramer staining. As shown in FIG. 1B, animals that mounted reduced immune response after the first therapeutic cycle, were less prone to develop substantial immune responses after the second therapeutic cycle (the animal model comprises HHD transgenic mice expressing variable levels of a human A2 allele of MHC class I—resulting in a situation that is reminiscent of inter-subject variability encountered in outbread populations such as human).

### Example 2

### Correlation Between the Magnitude of Immune Response as Measured by Tetramer Staining and Clearance of Human Tumor Cells in Vivo

HHD transgenic mice (n=4/group) were immunized with Melan A peptide, by direct inoculation into the inguinal lymph nodes with 25 μg in 25 μl of PBS/lymph node, with or without CpG or dsRNA adjuvant (12.5 μg admixed with peptide), at day 0 and 3. This was followed by two additional peptide boosts (similar amount, protocol and adjuvant) at day 28 and 31. The immune response was measured by tetramer staining using PBMC and HLA-A2-Melan A reagents (Beckman Coulter).

The mice were challenged with 624.38 melanoma cells (A2⁺, melan A⁺) stained with CFSE^{hi} fluorescence (4.0 μM for 15 minutes), co-injected into immunized mice with an equal ratio of 624.28 melanoma control cells (A2⁻, melan A⁺) stained with CFSE^{lo} fluorescence (0.4 μM). Eighteen hours later the specific elimination of target cells was measured by removing lung from challenged animals and measuring CFSE fluorescence by flow cytometry.

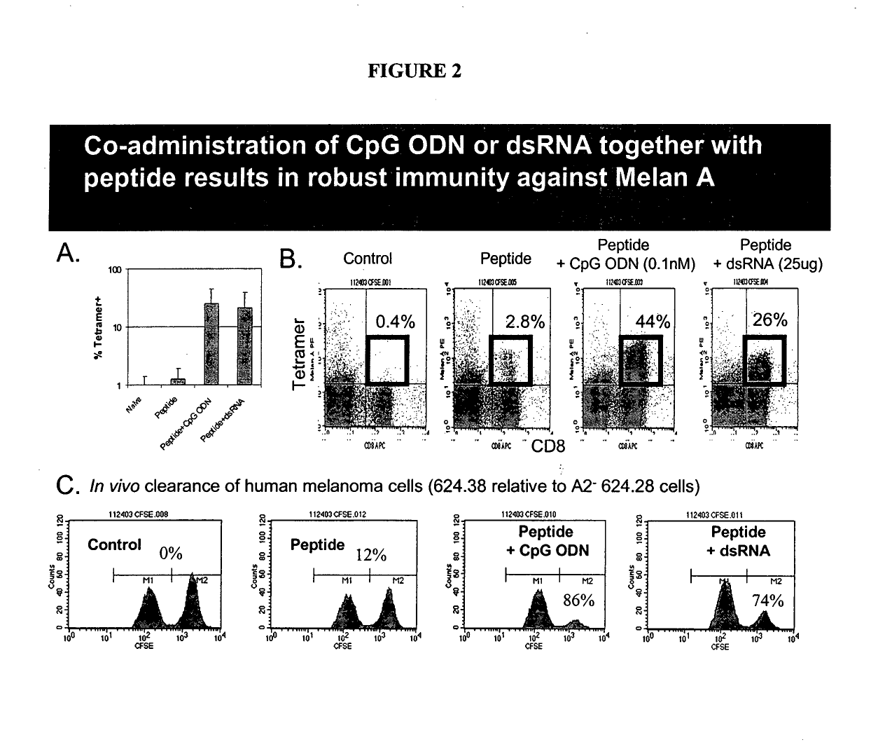

A direct correlation between the frequency of antigen specific T cells and the capability of immunized mice to clear human tumor cells over a defined interval of time was observed. Representative results are shown in FIG. 2. A frequency of specific T cells, in excess of 2% out of the CD8+ T cell population, was required for a significant clearance of the target cell population over that particular period of time.

### Example 3

### A Multivalent Immune Response (as Measured by ELISPOT Analysis) is Associated with in Vivo Clearance of Human Tumor Cells, in Contrast with a Monovalent Response

HHD transgenic mice (n=6) were immunized with a mixture of two plasmids (PSEM and pBPL, previously disclosed as pMA2M and pBPL, respectively, in U.S. patent application Ser. No. 10/292,413 entitled EXPRESSION VECTORS ENCODING EPITOPES OF TARGET-ASSOCIATED ANTIGENS AND METHODS FOR THEIR DESIGN, which is incorporated herein by reference in its entirety) expressing Tyrosinase 369-377, Melan A 26-35(A27L), SSX-2 41-49 and NY-ESO-1 157-165 epitopes, by direct inoculation into the inguinal lymph nodes (25 μg in 25 μl of PBS/lymph node) at day 1, 4, 15 and 18. This was followed by boosting doses of four peptide at day 28, 32, 49 and 53, of SSX-2 41-49 and Tyrosinase 369-377 peptide analogues into the left and right inguinal lymph nodes, respectively, (25 μg in 25 μl of PBS/lymph node).

The mice were challenged with 624.38 melanoma cells (A2⁺ melan A⁺) stained with CFSE^{hi} fluorescence (4.0μM for 15 minutes), co-injected into immunized mice with an equal ratio of 624.28 melanoma control cells (A2⁻, melan A⁺) stained with CFSE^{lo} fluorescence (0.4 μM). Eighteen hours later the specific elimination of target cells was measured by removing lung from challenged animals and measuring CFSE fluorescence by flow cytometry. In addition, the immune reactivity against each antigen was determined by ELISPOT analysis. In brief, various numbers of splenocytes were stimulated with 10 μg/ml of native peptides, separately, in ELISPOT plates coated with anti-IFN-γ antibody. After a 48-hour incubation, the assay was developed and the frequency of cytokine-producing T cells measured using a conventional biotin-streptavidin HRP assay. The data were expressed as number of spot forming colonies (mean of triplicates±SD) along with in vivo clearance of human tumor cells.

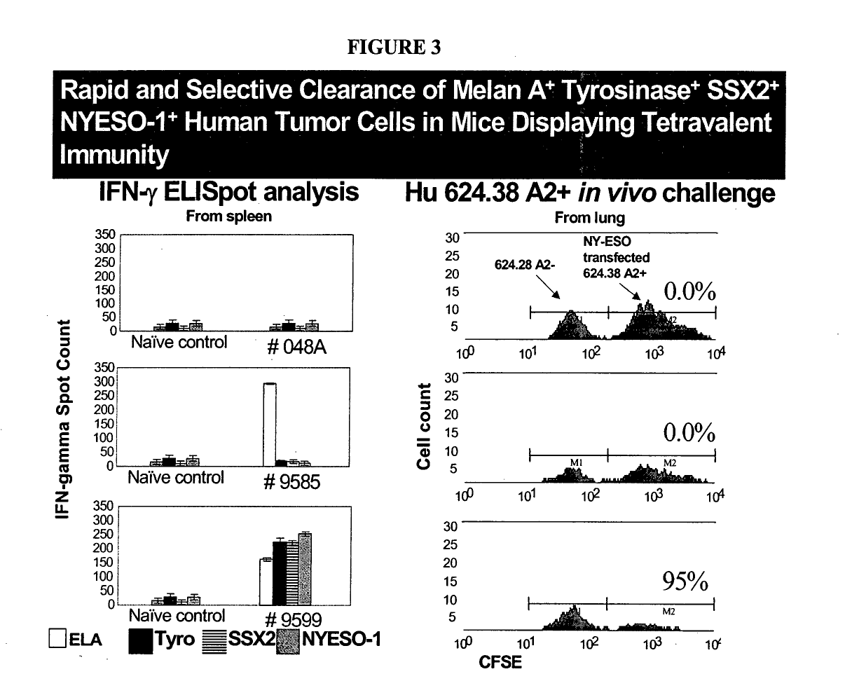

A multivalent response was associated with measurable clearance of human tumor cells over that particular interval, whereas the monovalent response, at the level of response achieved and over that time interval, was not associated with detectable clearance, as shown by the representative results in FIG. 3. This illustrates that methods of measuring the nature and specificity of immune response can be predictive for the biological effect; and thus, monitoring immune response can enable the effective adjustment or more radical modification of the therapeutic protocol.

### Example 4

### Immune Active Molecules can Have an Unexpected, Inverse Dose Effect Relationship (Increasing Dose, Diminishing Immune Response)

HHD transgenic mice (n=4/group) were immunized with a PRAME 425-433 expressing plasmid (as described in U.S. Provisional Application No. __/______ (Atty Docket No. MANNK.053PR), entitled METHODS AND COMPOSITIONS TO ELICIT MULTIVALENT IMMUNE RESPONSES AGAINST DOMINANT AND SUBDOMINANT EPITOPES, EXPRESSED ON CANCER CELLS AND TUMOR STROMA, filed on date even with this disclosure, which is hereby incorporated by reference in its entirety) or peptide together with dsRNA (different concentrations of plasmid or peptide, by direct inoculation into the inguinal lymph nodes with 25 μl of plasmid or peptide in PBS/lymph node, on days 0, 3, 14 and 18. Spleens were harvested at 10 days after completion of immunization, splenocytes were incubated over 48 hours with native peptide (10 μg/ml), supernatants harvested and IFN-gamma concentration measured by ELISA.

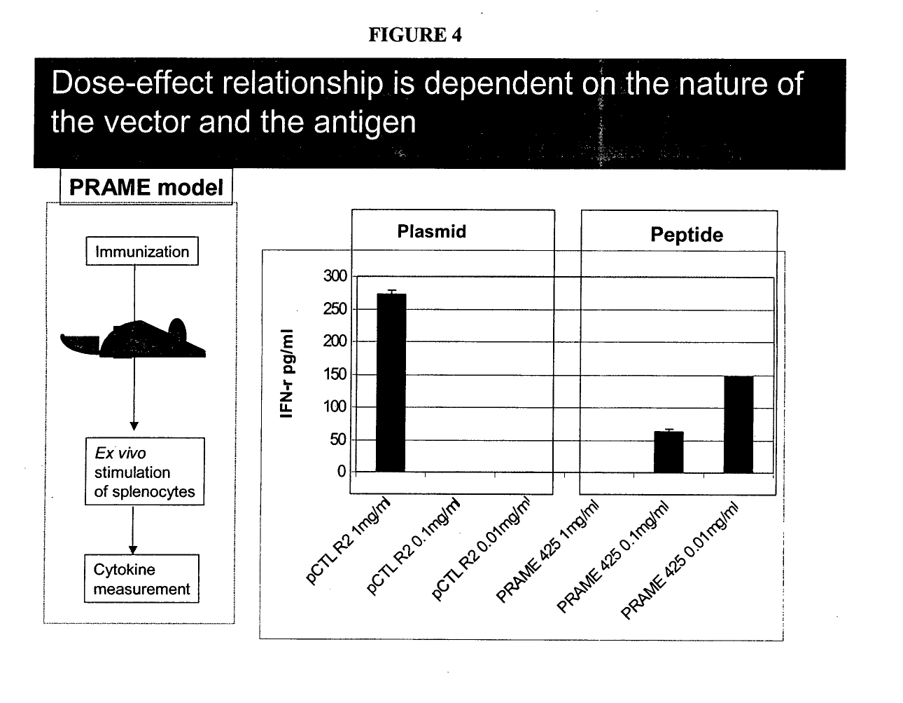

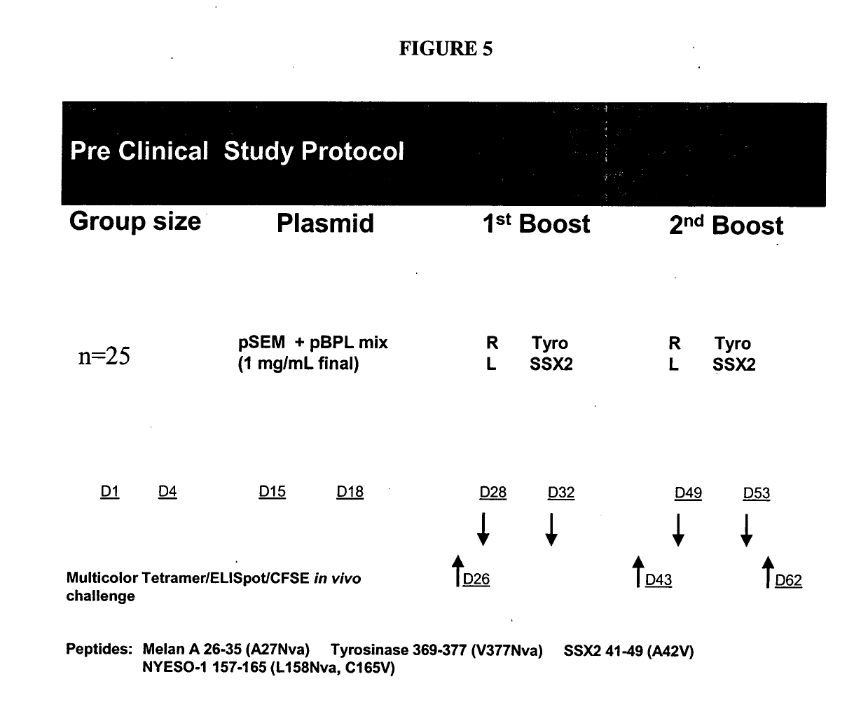

In this case it was observed that the larger the dose of plasmid, the greater the magnitude of the immune response. In contrast, the larger the dose of peptide, the lower the immune response (see FIG. 4; results expressed as pg IFN-gamma/ml). Thus, monitoring of immune response during a therapeutic regimen can be used to adjust the protocol of immunization, for example, to avoid high-zone tolerance phenomena as seen here.

### Example 5

### Protocol for Multivalent Priming Against Dominant and Subdominant Antigens Followed by Boost with the Subdominant Antigens

HHD transgenic mice were immunized with a mixture of two plasmids (pSEM and pBPL) expressing Tyrosinase 369-377, MelanA 26-35A27L, SSX-2 41-49 and NY-ESO-1 157-165 epitopes, by direct inoculation into the inguinal lymph nodes (25 μg in 25 μl of PBS/lymph node) at days 1, 4, 15 and 18. This was followed by boosting doses of peptide (similar amount) at days 28, 32, 49 and 53, of SSX-2 41-49 and Tyrosinase 369-377 peptide analogues into the left and right lymph nodes, respectively (25%1 g in 25 μl of PBS/lymph node), after interim determination of immune response (post-plasmid immunization phase and prior to boost with selected peptides). Additionally, Examples 6 and 7 below include naïve and plasmid boosted control groups.

### Example 6

### Heterogeneity of Immune Response in a Preclinical Model

HHD Transgenic Mice were Immunized as Described in Example 5

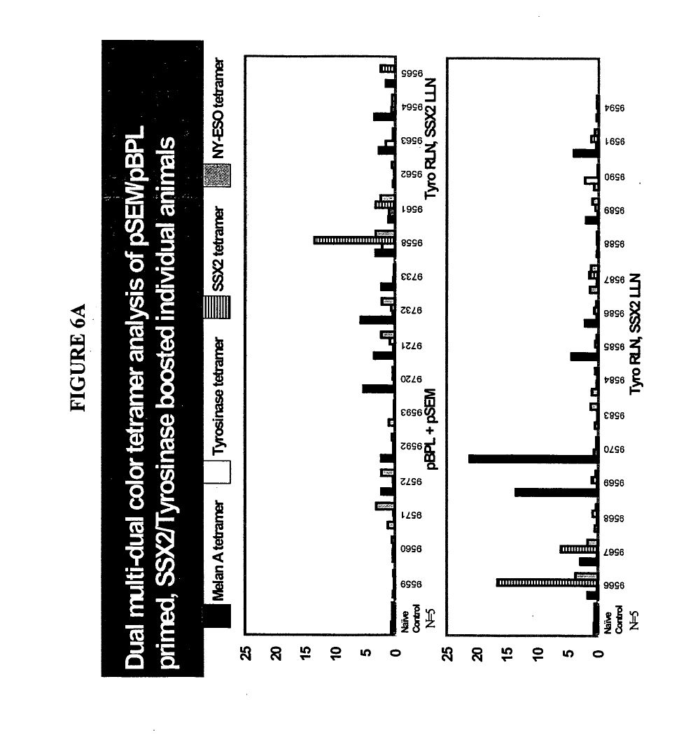

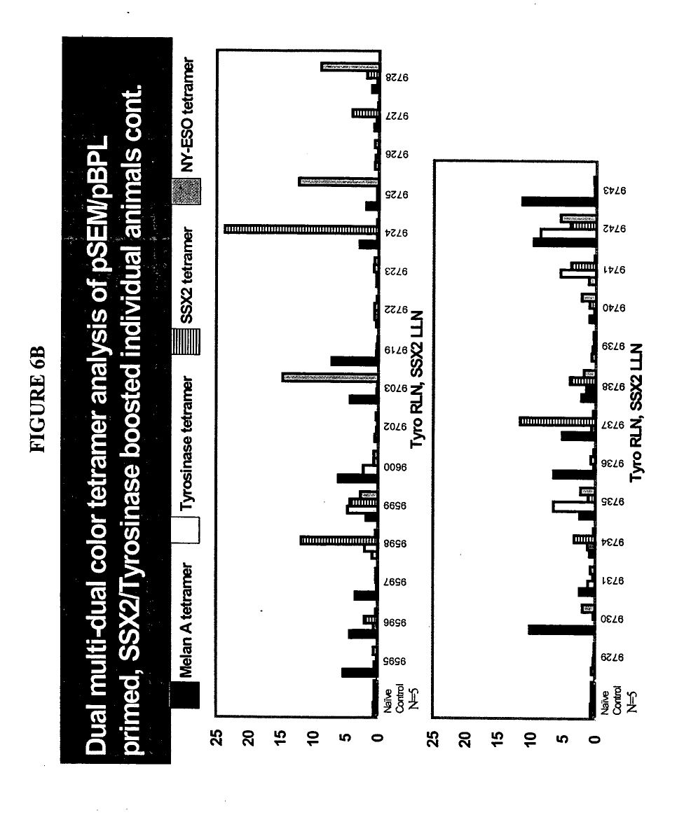

The immune reactivity against each antigen was determined by ELISPOT analysis. In brief, various numbers of splenocytes were stimulated with 10 μg/ml of native peptide, separately, in ELISPOT plates coated with anti-IFN-γ antibody. After a 48 hour incubation, the assay was developed and the frequency of cytokine-producing T cells measured using a conventional biotin-streptavidin HRP assay. The data were represented in FIGS. 6A and B as the number of spot forming colonies/well (mean of triplicates±SD).

The results showed significant heterogeneity from individual to individual as regards both the magnitude and antigen specificity of immune response. Thus it can be advantageous to monitor the immune reaction in order to adjust the protocol for the purpose of obtaining a balanced response that is more likely to elicit a therapeutic effect.

### Example 7

### Selective Boosting with Select Subdominant Epitopes, Results in a Balanced, Multivalent Immune Response

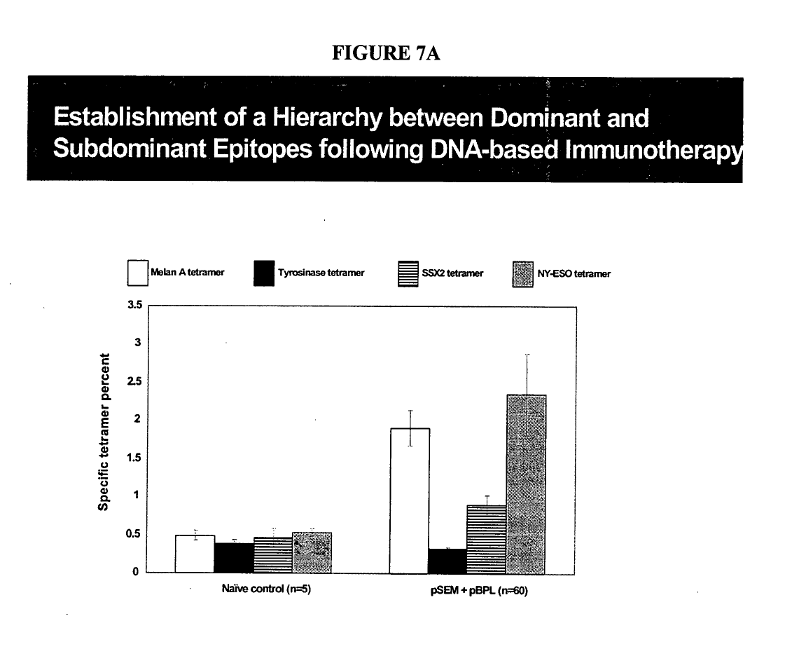

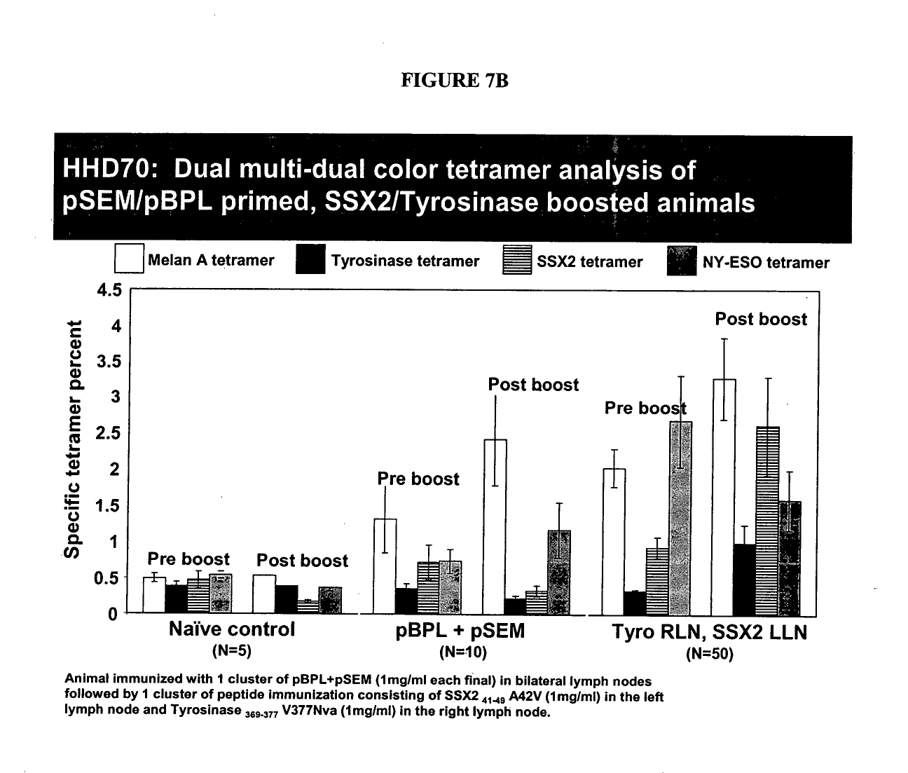

HHD transgenic mice were immunized as described in Example 5. Prior to administration of the peptide doses, measurement of specific response against each epitope was made using conventional tetramer staining (Beckman Coulter). The results in FIG. 7A, expressed as % tetramer reactive cells in the whole CD8⁺ T cell population (in blood), show that the immune response is dominated by T cells against Melan-A and NY-ESO-1, and the T cell populations against the other two antigens, SSX-2 and Tyrosinase, are comparatively small.

Consequently, this was followed by boosting doses of peptide (similar amount) at days 28, 32, 49 and 53, of SSX-2 41-49 and Tyrosinase 369-377 peptide analogues into the left and right lymph nodes, respectively (25 μg in 25 μl of PBS/lymph node). Measurement of immune response by tetramer staining showed a more balanced representation of all T cell populations in the mice boosted with the selected epitopes (FIG. 7B). Thus, a balanced response that is more likely to confer a clinical advantage may be achieved by adjusting a therapeutic regimen based on interim measurement of surrogate markers (such as immune response).

### Example 8

### Clinical Study Design Aimed at Analyzing the Relationship Between Immune Reactivity Against a Tumor Associated Antigen and Clinical Outlook

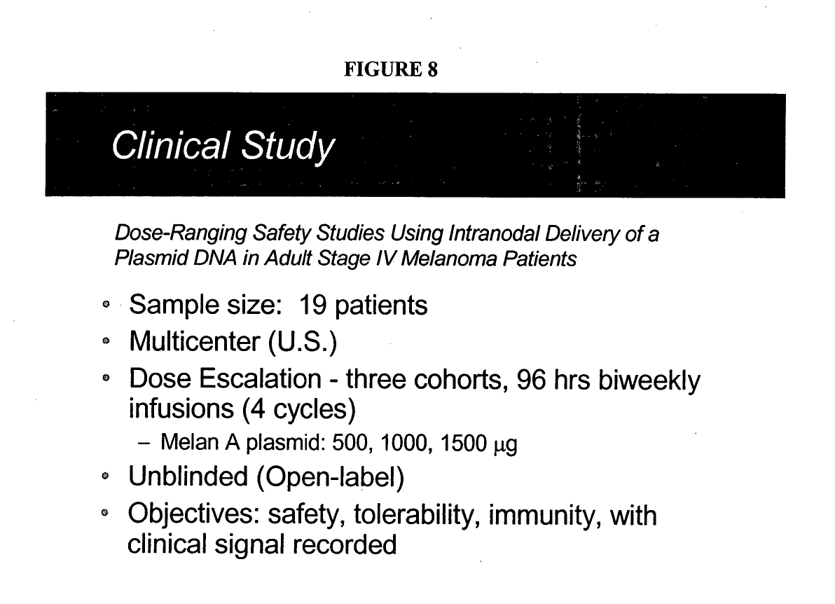

The immune response in stage IV melanoma patients was measured by tetramer staining before and after immunization by intra lymph node infusion with a plasmid (pSEM) expressing the MelanA 26-35A27L epitope (FIG. 8). In addition, the time to progression post-initiation of immune therapy, has been measured by conventional clinical means.

### Example 9

### Significant Relationship Between Immune Reactivity Against Melan A and Time to Disease Progression

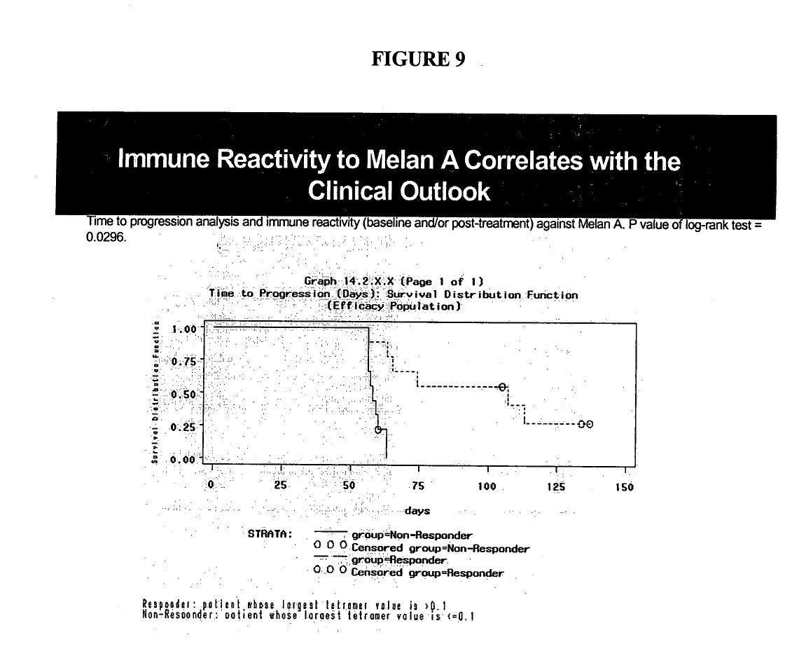

Measurement of immune reactivity (defined as frequency of MelanA-specific, tetramer+T cells in peripheral blood in excess of 0.1%) prior to and after immunization with pSEM plasmid (according to Example 8), showed a significant correlation (p<0.05) with the time-to-progression of disease. This illustrates the potential value in stratifying patient populations (good and poor prognosis) based on basal and treatment-induced immune reactivity (FIG. 9). The correlation between pre-treatment immune reactivity alone and clinical outlook, was not statistically significant (p>0.05).

### Example 10

### Clinical Application

An application of a methodology to diagnose “responders” versus “non-responders” by employing an immunization (“induction”) step that serves a dual diagnostic and therapeutic purpose.

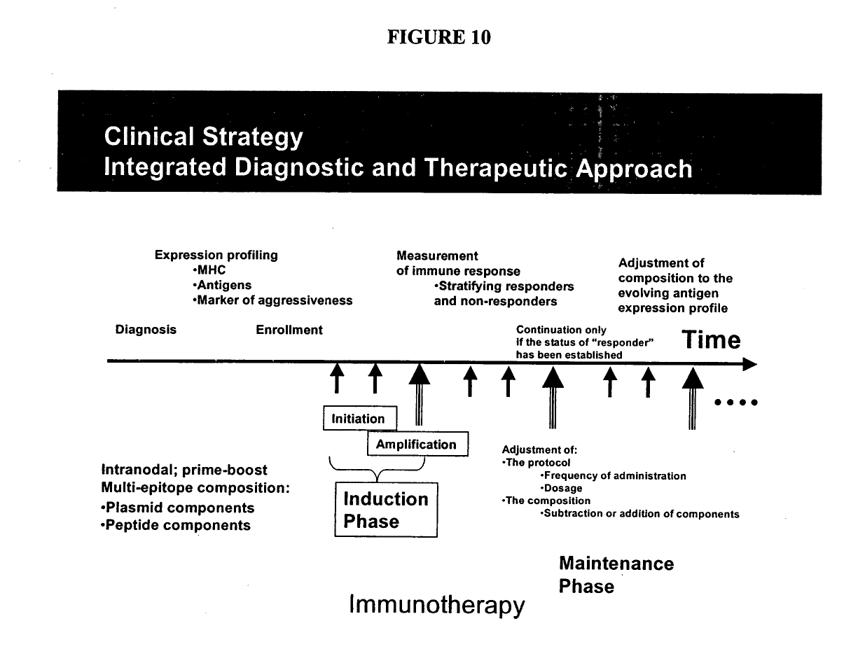

FIG. 10 provides a schema for integrated diagnostic and therapeutic clinical practice. A subject diagnosed with a disease is further examined for antigen and MHC expression on the target cells. Markers for the aggressiveness of the disease are also assessed. On this basis, if the subject is a candidate for treatment by active immunotherapy, immunization is initiated. General immune responsiveness, for example, by exposure to a toxoid, is not required. After an initial cycle of treatment, the subject is tested for an antigen specific response to the administered immunogen. Treatment is discontinued for non-responders.

For two-phase immunization protocols, immune alternate embodiments allow for testing responsiveness prior to administration of the second dosage form (intralymphatic peptide in FIG. 10). The first phase dosing regimen (plasmid in FIG. 10) is repeated before classifying subjects as “non-responders.” During the maintenance phase of treatment antigen expression of target cells is reassessed and the frequency of administration or the composition of the therapeutic is adjusted to match.

### Example 11

The methods disclosed herein are applied to an induce-and-amplify immunotherapy protocol that utilizes immunogenic compositions comprising distinct forms of immunogen for the two stages of the protocol. The protocol calls for four (4) cycles of six (6) inducing doses comprising a nucleic acid encoding the target antigen or a portion thereof followed by three (3) amplifying doses of comprising a targeted epitope of the antigen.

Briefly, 12 patients are each administered a plasmid as part of the third inducing dose of the protocol. Following administration of the plasmid, a sample of blood is taken from each patient and assayed to determine whether an immune response has occurred and how robust it is.

For seven patients, a measurable immune response is observed. The patients are tagged as “responders,” and the fourth inducing dose is administered according to protocol. For three patients, a minimal immune response is observed. The patients are tagged as “low responders.” For two of the three low responders, the third inducing dose is repeated, one at the dosage called for in the protocol, and the other at a higher dosage. For the third patient, the fourth inducing dose is administered at a higher dosage. For the remaining two patients, no immune response is observed. The patients are tagged as “non-responders.” For one of the two non-responders, treatment is discontinued and the patient is referred to an alternate therapy. For the second non-responder, the third inducing dose is repeated at a higher dosage.

Following the third complete cycle of inducing and amplifying doses, a sample of blood is taken from each remaining patient and assayed to determine whether an immune response has occurred and how robust it is.

For two of the patients a measurable immune response is observed. The patients are tagged as “high responders,” and the patients are administered further amplifying doses to maintain the response. For five of the patients, a minimal immune response is observed. The patients are tagged as “low-responders” and are administered additional complete cycles of induction and amplification. For two of the patients, no immune response is observed. The patients are tagged as “non-responders” and treatment is discontinued.

### Example 12

If the active immunotherapeutic is multivalent the assessment of responder status can be carried out in respect to each targeted antigen or epitope. Depending on the valency and immunodominance of the individual components of the immunogenic compositions, additional inducing or amplifying doses of any components eliciting lower responses can be re-administered in order to obtain a more balanced response to the various target antigens. Similarly, the relative dosages of the various components can be adjusted.

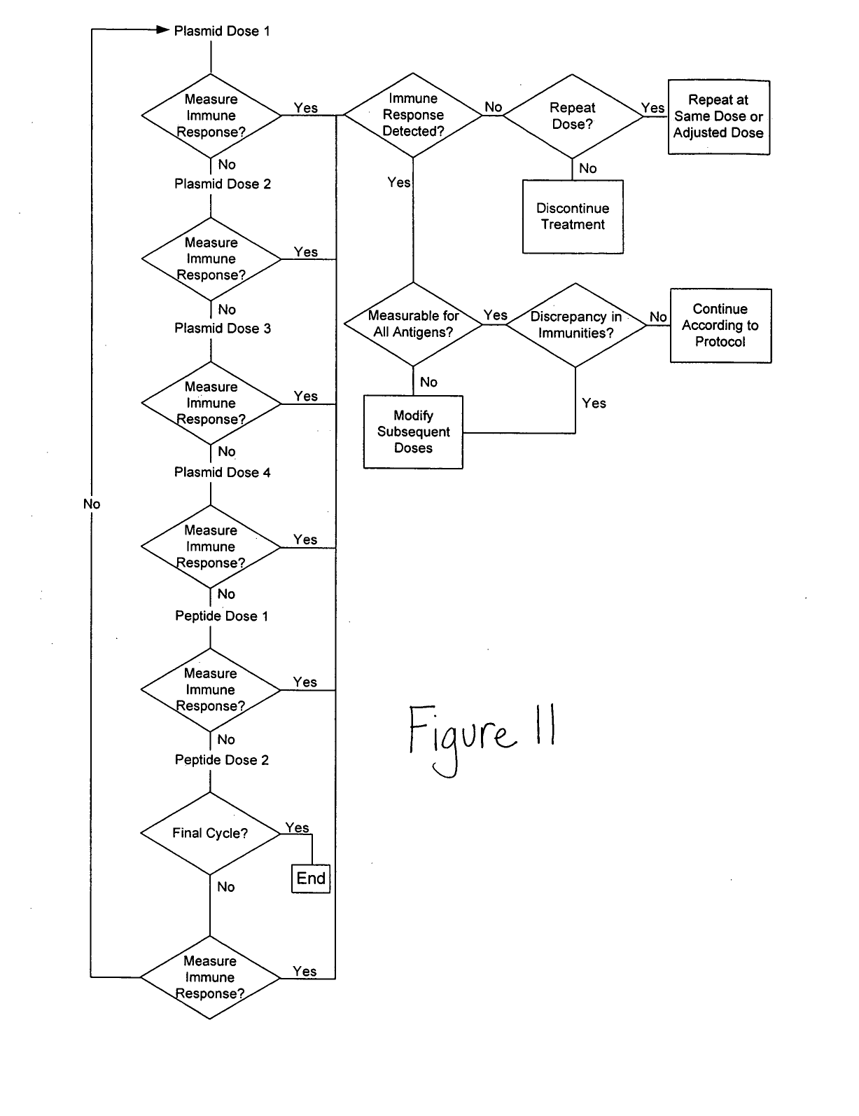

FIG. 11 provides an example of how the general principles described herein can be applied to a particular protocol.

Briefly, the protocol is as follows:

Induction phase: Induction of immunity by repeat plasmid administration into inguinal lymph nodes, at days 1, 4, 15, 18 (bilateral, bolus injection, 0.3 ml/bolus, 1.2 mg of plasmid/bolus, corresponding to 0.035 mg/kg). Dose range can be 0.0001-0.04 mg/kg of plasmid.

Amplification phase: Amplification of immunity by peptide injection on days 29 and 32 (bilateral, bolus injection, 0.3 ml/bolus, 0.3 mg of peptide/bolus, corresponding to 0.09 mg/kg; dose range can be 0.0001-0.1 mg/kg of peptide), for a total of four peptide injections per therapeutic cycle. Up to four different peptides can be accommodated (essentially, only one peptide will be delivered in a given bolus).

The protocol calls for the induction and amplification phases to be repeated at least two times.

According to the methods embodied herein, an immune response against the immunizing antigens is measured after any non-final dose of the protocol above.

If no immune response is detected, then the therapy is terminated and the patient referred to other types of therapies.

If the immune response is measurable against all antigens without significant discrepancies among them, then treatment is continued according to protocol.

If the immune response is nil against a first antigen and measurable against a second antigen, the protocol is modified, for example, immunization against the first antigen is discontinued and the therapeutic composition modified accordingly (to retain active eliciting immunity against the second antigen).

If the immunity against both antigens is significant, measurable, but immunity against a first antigen is substantially higher than immunity against a second antigen (discrepancy), then the therapeutic composition/regimen is modified, for example, to include additional boosts with actives amplifying response against the second antigen (in the form of peptide, e.g., administered at day 46 and 49, bilateral, bolus injection, 0.3 ml/bolus, 0.3 mg of peptide/bolus, corresponding to 0.09 mg/kg; dose range can be 0.0001-0.1 mg/kg of peptide) prior to resuming treatment.

1. A method for determining a course of treatment, wherein said treatment is based upon an immunotherapy protocol having multiple steps, said method comprising sequentially the steps of:
administering to a patient an immunogenic composition as part of a non-final step of the protocol;
measuring an immune response in said patient subsequent to the non-final step;
selecting a subsequent treatment action based on said measurement.

2. The method of claim 1, wherein the measuring step indicates no immune response, and the selecting step comprises discontinuation of the immunotherapy protocol.

3. The method of claim 1, wherein the measuring step indicates a minimal immune response and the selecting step comprises repeating the non-final dose of the protocol

4. The method of claim 1, wherein the measuring step indicates a substantial immune response and the selecting step comprises administering a second immunogenic composition.

5. The method of claim 4, wherein the immunogenic compositions are provided in a form selected from the group consisting of DNA, mRNA, plasmid, peptide, polypeptide, protein, viral vector, virus-like particle, and bacterial vector.

6. The method of claim 4, wherein the first and second immunogenic compositions are provided in a form that is the same.

7. The method of claim 4, wherein the first and subsequent immunogenic compositions are provided in forms that are different.

8. The method of claim 1, wherein the immunogenic composition comprises a target-matched immunogen.

9. The method of claim 8, wherein the immunogen comprises an antigen or a portion thereof.

10. The method of claim 9, wherein the immunogenic composition further comprises an immunopotentiating agent.

11. The method of claim 8, wherein the immunogen comprises a nucleic acid encoding the antigen or portion thereof.

12. The method of claim 1, wherein the immunogenic composition is multivalent.

13. The method of claim 1, wherein the measurement step comprises an assay selected from the group consisting of a cytokine assay, a cell proliferation assay, an Elispot assay, a cytotoxicity assay, a chromium release assay, an immunofluorescence assay, an MHC-multimer assay, a flow cytometry assay, an antibody assay, a DTH assay, and an inflammatory reaction assay.

14. The method of claim 1, wherein the subsequent treatment action comprises an action selected from the group consisting of: administering a predetermined subsequent dose of the protocol, administering a subsequent dose at an increased dosage, administering a subsequent dose at a decreased dosage, administering subsequent doses more frequently, administering subsequent doses less frequently, repeating administration of said preceding dose, selectively administering individual components of the composition, selectively suspending administration of individual components of the composition, and discontinuing treatment prior to completion of the protocol.

15. A method of treating a patient comprising sequentially the steps of:
administering to the patient an immunogenic composition as part of a non-final step of a multi-step immunization protocol;
assaying a patient sample for immune responsiveness to a component of the composition subsequent to the non-final step;
classifying the patient as a responder, a low-responder, or a non-responder based on the immune responsiveness; and
selecting a subsequent treatment action based on the classification.

16. The method of claim 15, wherein the classifying step comprises classifying the patient as a non-responder and the selecting step comprising discontinuing treatment.

17. The method of claim 15, wherein the non-final dose is an inducing dose of an induce-and-amplify protocol.

18. The method of claim 15, wherein the classifying step comprises classifying the patient as a low-responder and the selecting step comprising administering an additional inducing dose.

19. The method of claim 15, wherein the classifying step comprises classifying the patient as a responder and the selecting step comprising administering an amplifying dose.

20. The method of claim 15, wherein the immunogenic composition comprises a composition targeting at least two target antigens.

21. The method of claim 20, wherein the assaying step comprises determining immune responsiveness to at least two target antigens.

22. The method of claim 21, wherein the classifying step comprising classifying the patient as a responder with respect to a first target antigen and a low-responder with respect to a second target antigen, and the selecting step comprises administering an immunogenic composition comprising a component corresponding to the second target antigen, but not to the first target antigen.

23. A method of treating a patient comprising the steps of:
administering to the patient an immunogenic composition as part of a non-final step of a multi-step immunotherapy protocol; wherein the immunogenic composition targets one or more antigens;
assaying tumor tissue from the patient for expression of the one or more antigens subsequent to the non-final step;
establishing an antigen expression profile; and
optimizing the match between the expression profile and the one or more antigens targeted by the immunogenic composition.
